SEQUENCE LISTING (25 sequences)

SEQ ID NO: 1 - protein, 28 aa, Homo sapiens
  relaxin-1 b-chain
KWKDDVIKLCGRELVRAQIAICGMSTWS

SEQ ID NO: 2 - protein, 29 aa, Homo sapiens
  relaxin-2 b-chain
DSWMEEVIKLCGRELVRAQIAICGMSTWS

SEQ ID NO: 3 - protein, 26 aa, Homo sapiens
  relaxin-3 b-chain
RAAPYGVRLCGREFIRAVIFTCGGRW

SEQ ID NO: 4 - protein, 30 aa, Homo sapiens
  insulin b-chain
FVNQHLCGSHLVEALYLVCGERGFFYTPKT

SEQ ID NO: 5 - protein, 29 aa, Homo sapiens
  insulin-like growth factor 1 (IGF-1) b-chain
GPETLCGAELVDALQFVCGDRGFYFNKPT

SEQ ID NO: 6 - protein, 31 aa, Homo sapiens
  insulin-like growth factor 2 (IGF-2) b-chain
YRPSETLCGGELVDTLQFVCGDRGFYFSRPA

SEQ ID NO: 7 - protein, 31 aa, Homo sapiens
  insulin-like 3 (INSL3) b-chain
PTPEMREKLCGHHFVRALVRVCGGPRWSTEA

SEQ ID NO: 8 - protein, 33 aa, Homo sapiens
  insulin-like 4 (INSL4) b-chain
ESLAAELRGCGPRFGKHLLSYCPMPEKTFTTTP

SEQ ID NO: 9 - protein, 33 aa, Homo sapiens
  insulin-like 5 (INSL5) b-chain
VRSKESVRLCGLEYIRTVIYICASSRWRRHLEG

SEQ ID NO: 10 - protein, 33 aa, Homo sapiens
  insulin-like 6 (INSL6) b-chain
SDISSARKLCGRYLVKEIEKLCGHANWSQFRFE

SEQ ID NO: 11 - protein, 25 aa, Artificial Sequence
  Description of Artificial Sequencecyclic relaxin b-chain mimetic (cRlx)
  DISULFID (2)..(24)
SCMEEVIKLSGRELVRAQIAISGCS

SEQ ID NO: 12 - protein, 27 aa, Artificial Sequence
  Description of Artificial Sequenceinsulin-like 3 (INSL3) b-chain peptide analogue 4, cyclic peptide cINSL3a
  DISULFID (3)..(25)
TPCMREKLSGHHFVRALVRVSGGPCWS

SEQ ID NO: 13 - protein, 27 aa, Artificial Sequence
  Description of Artificial Sequenceinsulin-like 3 (INSL3) b-chain peptide analogue 5, cyclic peptide cINSL3b
  DISULFID (3)..(25)
TPCMREKLSGRHFVRALVRVSGGPCWS

SEQ ID NO: 14 - protein, 27 aa, Artificial Sequence
  Description of Artificial Sequenceinsulin-like 3 (INSL3) b-chain peptide analogue 6
TPCMREKLSGRELVRAQVIAIGGPCWS

SEQ ID NO: 15 - protein, 27 aa, Artificial Sequence
  Description of Artificial Sequenceinsulin-like 3 (INSL3) b-chain peptide analogue 7
TCEMREKLSGHHFVRALVRVSGGCRWS

SEQ ID NO: 16 - protein, 24 aa, Homo sapiens
  relaxin-1 a-chain
RPYVALFEKCCLIGCTKRSLAKYC

SEQ ID NO: 17 - protein, 24 aa, Homo sapiens
  relaxin-2 a-chain
QLYSALANKCCHVGCTKRSLARFC

SEQ ID NO: 18 - protein, 24 aa, Homo sapiens
  relaxin-3 a-chain
DVLAGLSSSCCKWGCSKSEISSLC

SEQ ID NO: 19 - protein, 26 aa, Homo sapiens
  insulin a-chain
SLQKRGIVEQCCTSICSLYQLENYCN

SEQ ID NO: 20 - protein, 25 aa, Homo sapiens
  insulin-like growth factor 1 (IGF-1) a-chain
APQTGIVDECCFRSCDLRRLEMYCA

SEQ ID NO: 21 - protein, 25 aa, Homo sapiens
  insulin-like growth factor 2 (IGF-2) a-chain
RRSRGIVEECCFRSCDLALLETLCA

SEQ ID NO: 22 - protein, 26 aa, Homo sapiens
  insulin-like 3 (INSL3) a-chain (Leydig insulin-like (Ley I-L)/relaxin like factor (RLF))
AAATNPARYCCLSGCTQQDLLTLCPY

SEQ ID NO: 23 - protein, 25 aa, Homo sapiens
  insulin-like 4 (INSL4) a-chain (placentin/early placenta insulin-like (EPIL))
RSGRHRFDPFCCEVICDDGTSVKLC

SEQ ID NO: 24 - protein, 24 aa, Homo sapiens
  insulin-like 5 (INSL5) a-chain
MSRQDLQTLCCTDGCSMTDLSALC

SEQ ID NO: 25 - protein, 24 aa, Homo sapiens
  insulin-like 6 (INSL6) a-chain
RKRRGYSEKCCLTGCTKEELSIAC

(19) United States
(12) Patent Application Publication
(10) Pub. No.: US 2007/0004619 A1
(43) Pub. Date: Jan. 4, 2007

(54) Relaxin superfamily peptide analogues

(75) Inventors: Mark Del Borgo, Parkville (AU); John D. Wade, Parkville (AU); Ross D. Bathgate, Parkville (AU); Richard A. Hughes, Parkville (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Howard Florey Institute of Experimental Physiology and Medicine, Victoria (AU)

(21) Appl. No.: 10/561,304

(22) PCT Filed: Jun. 18, 2004
(86) PCT No.: PCT/AU04/00798

§ 371 Date: Jun. 19, 2006

(30) Foreign Application Priority Data
Jun. 20, 2003 (AU) 2003903124

(51) Int. Cl. A61K 38/22

(52) U.S. Cl. 514/9

(57) ABSTRACT

Monomeric, cyclic peptide analogues of relaxin superfamily proteins, based on B-chains of relaxin superfamily proteins, and methods of making them, are described, as well as pharmaceutical compositions containing them and methods of utilising them.

### FIELD OF THE INVENTION

This invention relates to generally to conformationally constrained, monomeric peptide analogues of the relaxin superfamily of heteromeric proteins and in particular, to monomeric cyclic analogues of the B-chain of relaxin superfamily proteins, methods of making these analogues, and uses thereof.

### BACKGROUND OF THE INVENTION

The relaxin superfamily currently comprises 10 members with a relatively high degree of sequence homology. These family members include insulin, insulin-like grown factors I and II, relaxin 1, 2 and 3, and the insulin-like hormones INSL3, 4, 5 and 6. The relaxin superfamily members have a wide range of biological activities which are well described in the art.

The critical action of insulin has been described for decades. Insulin has a key role in cellular metabolism. Many subjects with diabetes require daily administration of insulin.

Relaxin is an insulin-like peptide having two separate chains (A and B) joined by two interchain and one intrachain disulfide bond. The actions of relaxin include an ability to inhibit myometrial contractions, stimulation of remodelling of connective tissue and induction of softening of the tissues of the birth canal. Additionally, relaxin increases growth and differentiation of the mammary gland and nipple and induces the breakdown of collagen, one of the main components of connective tissue. Relaxin decreases collagen synthesis and increases the release of collagenases (Unemori et al (1990) J. Biol. Chem. 265, 10682-10685). These findings were recently confirmed by the establishment of the relaxin gene-knockout mouse (Zhao et al (1999) Endocrinology 140, 445-453), which exhibited a number of phenotypic properties associated with pregnancy. Female mice lacking a functionally active relaxin gene failed to relax and elongate the interpubic ligament of the pubic symphysis and could not suckle their pups, which in turn, died within 24 hours unless cross-fostered to relaxin wild type or relaxin heterozygous foster mothers.

Evidence has accumulated to suggest that relaxin is more than a hormone of pregnancy and acts on cells and tissues other than those of the female reproductive system. Relaxin causes a widening of blood vessels (vasodilatation) in the kidney, mesocaecum, lung and peripheral vasculature, which leads to increased blood flow or perfusion rates in these tissues (Bani et al (1997) Gen. Pharmacol. 28, 13-22). It also stimulates an increase in heart rate and coronary blood flow, and increases both glomerular filtration rate and renal plasma flow (Bani et al (1997) Gen. Pharmacol. 28, 13-22). Relaxin has also been found to inhibit histamine release and the accumulation of calcium, as well as promote nitric oxide synthesis, during cardiac anaphylaxis (Masini et al., Br J. Pharmacol. 2002 October; 137(3):337-44). The brain is another target tissue for relaxin where the peptide has been shown to bind to receptors (Osheroff et al (1991) Proc. Nal. Acad. Sci. U.S.A. 88, 6413-6417; Tan et al (1999) Br. J. Pharmacol 127, 91-98) in the circumventricular organs to affect blood pressure and drinking (Parry et al (1990) J Neuroendocrinol. 2, 53-58; Summerlee et al (1998) Endocrinology 139, 2322-2328; Sinnahay et al (1999) Endocrinology 140, 5082-5086).

Important clinical uses arise for relaxin in various diseases responding to fibrotic breakdown, connective tissue remodelling, scleroderma, vasodilation, such as coronary artery disease, peripheral vascular disease, kidney disease associated with arteriosclerosis or other narrowing of kidney capillaries, or other capillaries narrowing in the body, such as in the eyes or in the peripheral digits, the mesocaecum, lung and peripheral vasculature, and neurological modification. Relaxin has also been implicated in depression of platelet aggregation and their release by megakaryocytes, and may thus be associated with clotting disorders.

The INSL peptides, such as INSL3, may be involved in various physiological actions, including descent of the gonads. In particular, INSL3 has been shown to be involved in maturation and descent of the testes (Ivell & Hartnung, Mol Hum Reprod 2003, 9(4): 175-181), as well as the survival of sperm cells, development of ovarian follicles and maturation of the oocyte (Kawamura et al., 2004, PNAS 101, 7323-7328). Therefore, potential clinical applications of INSL3 agonists and antagonists include the treatment of fertility disorders, or the control of fertility levels.

INSL3 has been implicated in regulation of relaxin activity in the heart (Tan et al., Eur J Pharmacol. 2002 Dec. 20; 457(2-3):153-60), and may thus play an important regulatory role in cardiovascular disease.

Furthermore, INSL3 polymorphisms have been hyperplastic and neoplastic disorders of the thyroid gland (Hombach-Klonisch et al., Int J Oncol 2003 May; 22(5): 993-1001), suggesting a role for this relaxin superfamily member in the etiology of these pathologies.

The biological actions of the relaxin superfamily members are mediated through hormone receptors. In particular, the insulin receptor, the IGF-I and IGF-II receptors, GPCR135, GPCR142, as well as LGR7 and LGR8. LGR7 is the relaxin receptor, which binds all human relaxin peptides with high affinity, but has very low affinity for INSL3 (Bathgate et al., Trends in Endocrinology and Metabolism 2003, 14: 207-213). LGR8 is the INSL3 receptor, which binds INSL3 with high affinity. Knockout mice which lack a LGR8 gene have identical phenotype to the INSL3 knockout mice. However in the human, LGR8 will also bind relaxin, although it has a higher affinity for INSL3 (Bathgate et al., Trends in Endocrinology and Metabolism 2003, 14: 207-213).

The LGR8 receptor is expressed in the gubernaculum, ovary and testis, where in the latter organ it mediates INSL3's actions on testes descent (Bathgate et al., Trends in Endocrinology and Metabolism 2003, 14: 207-213). In the ovary, LGR8 mediates follicular development and in both the testes and ovaries, it is implicated in male and female germ-cell maturation (Kawamura et al., 2004, PNAS 101, 7323-7328). LGR8 is also expressed in the kidney, bone, peripheral-blood leucocytes, muscle, brain, thyroid, and uterus (Hsu et al., Science, 295: 671-674). Hence, INSL3 has potential actions on kidney and thyroid function, central actions on the brain, modulation of muscle function, effects on bone and blood cell function and reproductive actions on the uterus.

There is a need for analogues that are relaxin-superfamily analogues which bind at the biological receptors for the relaxin superfamily proteins. Ideally, such analogues would include ligands, such as agonists, reverse agonists, partial agonists, mixed agonists/antagonists and full antagonists, which bind at the relaxin superfamily member receptors and initiate, inhibit, activate, or otherwise control, the biological activities of members of this protein superfamily. Analogues of the relaxin superfamily proteins could provide the opportunity for a wide range of therapeutic interventions.

### SUMMARY OF THE INVENTION

In its broadest form, the present invention relates to peptide analogues of members of the relaxin superfamily of proteins, the analogues having a binding activity at a relaxin protein superfamily receptor, methods of producing the analogues, and pharmaceutical compositions, methods of treatment, and uses of the peptide analogues.

In a first aspect, the analogue is a conformationally constrained, monomeric cyclic peptide analogue of a B-chain of a relaxin superfamily member protein which binds to a biological target of the relaxin superfamily protein, the protein being selected from insulin, IGF-I, IGF-II, relaxin 1, relaxin 2, relaxin 3, INSL3, INSL4, INSL5 or INSL6.

The monomeric peptide analogues of the invention are produced by modification of a turn or loop moiety of the B-chain of the relaxin superfamily protein, the modification involving selection of at least a first and a second amino acid residue with an alpha-helix or beta-strand carbon separation distance of less than six Angstroms and cross-linking the first and second amino acids, wherein the cross-link conformationally constrains the analogue.

In one aspect, the monomeric analogue is an INSL3 B-chain analogue modified from the sequence set forth in SEQ ID NO:7.

In a further aspect, the INSL3 analogue is constrained by a cross-link between a first amino acid within a range of positions 2 and 8 and a second amino acid within a range of positions 21 and 26 of the sequences set forth in SEQ ID NO:7.

In a particularly preferred form, the first and/or second amino acids are substituted with amino acids suitable for cross-linking.

According to another aspect, the monomeric analogue is the INSL3 analogue designated cINSLa in FIG. 3.

In a further aspect, the monomeric analogue is the INSL3 analogue designated cINSLb in FIG. 3.

In yet another aspect, the monomeric analogue is a relaxin analogue modified from a relaxin-1, relaxin-2, or relaxin-3 B-chain sequence set forth in SEQ ID NOs: 1, 2 and 3, respectively.

In a preferred form, the monomeric relaxin analogue is the relaxin analogue designated cR1x in FIG. 3.

In a further aspect, the monomeric relaxin analogue is constrained by a cross-link between a first amino acid within a range of positions 2 and 8 and a second amino acid within a range of positions 21 and 26 of the sequences set forth in SEQ ID NO:2.

In yet a further form, one or more amino acids within the INSL3 or relaxin peptide analogue sequence, other than the cross-linked first and second amino acids, is optionally substituted to modify one or more biological activities of the monomeric analogue.

In a further preferred form, the monomeric analogue is an agonist, partial agonist, antagonist, partial antagonist or reverse antagonist at the relaxin superfamily receptor.

In another aspect, the analogues of the invention are conjugated to an A-chain of the relaxin superfamily protein.

In a further aspect, the monomeric analogues of the invention are conjugated to carrier molecules, such as labelling compounds, radioligands, radionucleotides, immunotherapeutic agents or other desirable chemical moiety.

In a further aspect, the invention seeks to provide a method of making a monomeric peptide analogue of a relaxin superfamily protein, the method comprising:
providing a B-chain, receptor-binding subunit of a relaxin superfamily protein; and
identifying a first and a second amino acid on respective, opposing strands of a turn and/or loop moiety in the receptor-binding subunit of the protein, with an alpha-helix or beta-strand carbon separation distance of less than six angstroms; and
introducing a cross-link between the first and second amino acids;
   wherein the cross-link constrains a three dimensional structure of at the B-chain, receptor-binding subunit.

In a particularly preferred form, the first and/or second amino acids are substituted with amino acids suitable for cross linking.

In one aspect, the method produces an INSL3 peptide analogue based on the sequence set forth in SEQ ID NO:7.

In a preferred form, the method produces the INSL3 analogue designated cINSLa in FIG. 3.

In another preferred form, the method produces the INSL3 analogue designated cINSLb in FIG. 3.

In a further aspect, the method produces a monomeric relaxin-1, relaxin-2 or relaxin-3 peptide analogue based on any of the sequences set forth in SEQ ID NOs:1, 2 and 3, respectively.

Preferably, the method produces a monomeric relaxin analogue designated cR1x in FIG. 3.

In yet a further form, the method produces an INSL3 and/or relaxin analogue wherein one or more amino acids other than the cross-linked first and second amino acids, are optionally substituted to modify one or more biological activities of the peptide analogue.

In yet another aspect, there is provided a pharmaceutical composition including one or more of the monomeric analogues of the present invention, and pharmaceutically acceptable salts thereof.

Preferably, the pharmaceutical composition includes an INSL3 analogue or a relaxin analogue.

A method of treating, preventing or ameliorating the symptoms of a disorder in a patient, the disorder involving disregulation of activity of a relaxin superfamily protein member, the method comprising administering to the patient an effective amount of a peptide analogue of the invention.

In one aspect, the disorder is characterised by disregulation of relaxin and/or INSL3 expression or activity.

In another aspect, the invention provides a method of treating, preventing or ameliorating the symptoms of a relaxin or INSL3-mediated disorder in a patient, wherein the disorder is selected from the group consisting of hyperplastic disorders, neoplastic disorders, scleroderma, uncontrolled or abnormal collagen or fibronectin formation or breakdown, neurological disorders, angiogenic disorders, cardiovascular disorders, female reproductive disorders, conditions associated with pregnancy, renal disease, inflammatory bowel disease, Raynaud's disease, Raynaud's phenomenon, cryptorchidism, disregulation of spermatogenesis and reproductive development including descent of the gonads, the method comprising administering to the patient an effective amount of a peptide analogue of the invention.

In a further aspect, the invention contemplates the use of the analogues and/or pharmaceutical compositions of the present invention in the manufacture of a medicament for the treatment of a relaxin or INSL3-mediated disorder in a patient, the disorder selected from the group consisting of: hyperplastic disorders, neoplastic disorders, scleroderma, uncontrolled or abnormal collagen or fibronectin formation or breakdown, neurological disorders, angiogenic disorders, cardiovascular disorders, female reproductive disorders, conditions associated with pregnancy, renal disease, inflammatory bowel disease, Raynaud's disease, Raynaud's phenomenon, cryptorchidism, disregulation of spermatogenesis and reproductive development including descent of the gonads.

### DESCRIPTION OF THE FIGURES

FIG. 1: Schematic representation of binding of INSL3, cINSLa and cINSLb analogues to the LGR8 receptor.

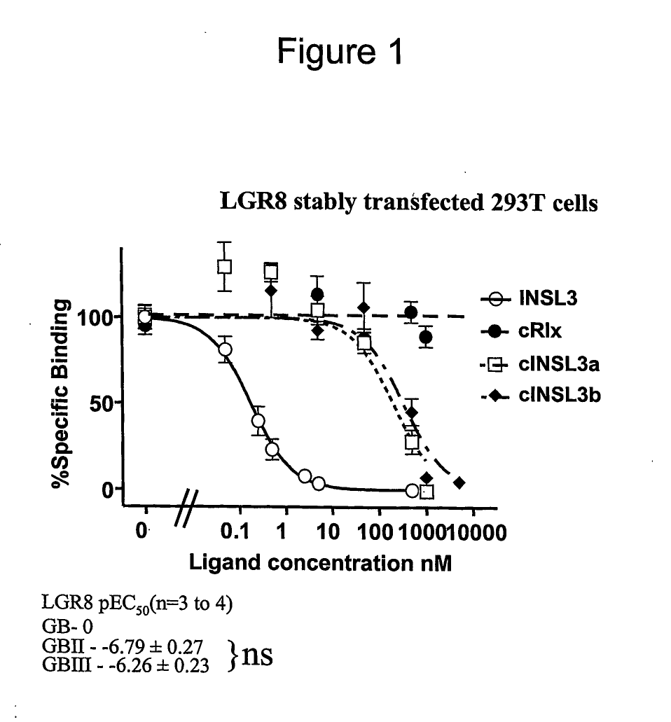

FIG. 2: Schematic representation of binding of the relaxin-like or INSL3 analogues to the LGR7 receptor.

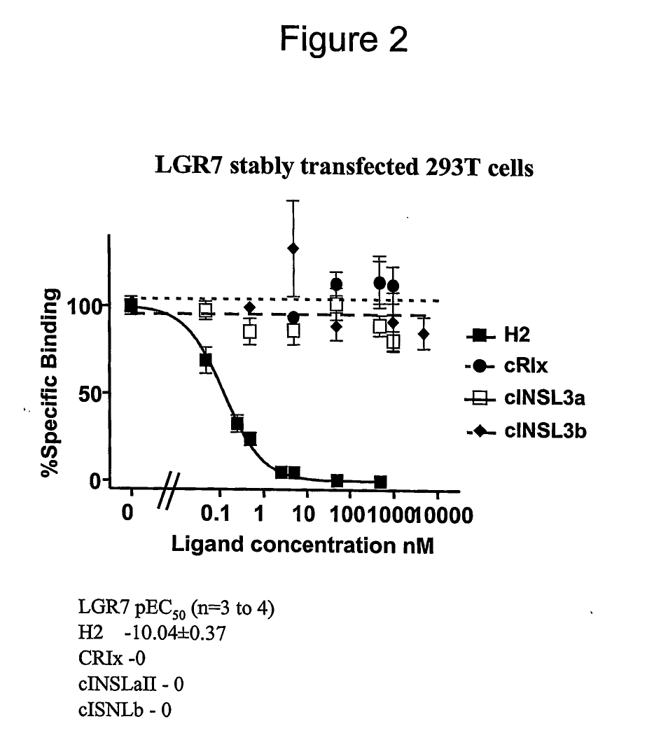

FIG. 3: Sequences and constraints of the native relaxin and INSL3-B chains with exemplary analogues designed.

FIG. 4: Schematic representation of antagonistic activity of cINSLa in the inhibition of the response of INSL3 (measured by cAMP response) to the native INSL3 receptor (LGR8).

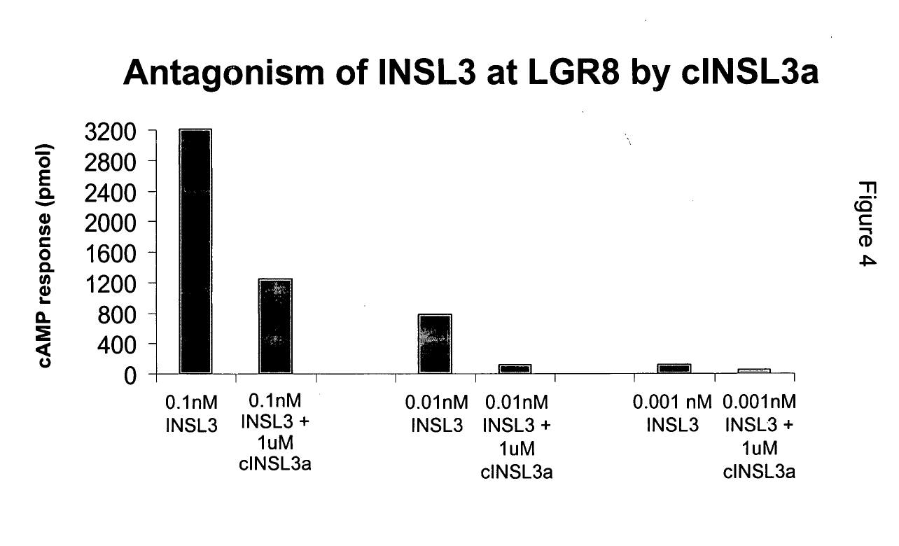

FIG. 5: Schematic representation illustrating CD spectra of cINSLa showing significant alpha-helical content in water and phosphate buffered saline.

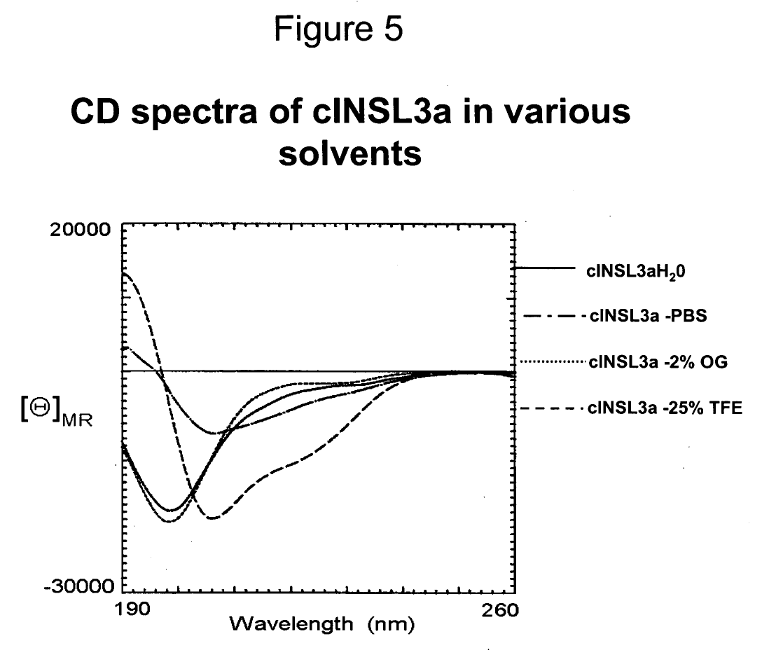

### DETAILED DESCRIPTION

In its broadest form, the present invention relates to peptide analogues of members of the relaxin superfamily of proteins, the analogues having a binding activity at a relaxin protein superfamily receptor, methods of producing the analogues, and pharmaceutical compositions, methods of treatment, and uses of the peptide analogues.

In a first aspect, the analogue is a conformationally constrained, cyclic analogue of a B-chain of a relaxin superfamily member protein which binds to a biological target of a relaxin superfamily protein, and modulates an activity of the biological target, wherein the relaxin superfamily protein is selected from insulin, IGF-I, IGF-II, relaxin 1, relaxin 2, relaxin 3, INSL3, INSL4, INSL5 or INSL6.

The term “modulates” as used herein includes, but is not limited to, initiation of activity at a biological target of a relaxin superfamily protein, up- or down-regulation of activity at a biological target of a relaxin superfamily protein, or blockage of an activity at a biological target of a relaxin superfamily protein.

The biological targets of relaxin superfamily proteins include receptors, such as but not limited to, LGR7, LGR8, GPCR135, GPCR142, the insulin receptor, the IGF-1 receptor, and the IGF-II receptor.

Accordingly, the analogue can also be a ligand at a relaxin superfamily receptor, wherein the analogue ligand can have an activity including, but not limited to, that of an agonist, antagonist, mixed agonist, reverse agonist, partial agonist, partial antagonist or reverse antagonist at the receptor.

The cyclic B-chain peptide analogues of the invention are produced by modification of a turn or loop moiety of the B-chain of the relaxin superfamily protein, the modification involving selection of at least a first and a second amino acid residue with an alpha-helix or beta-strand carbon separation distance of less than six Angstroms and cross-linking the first and second amino acids, wherein the cross-link conformationally constrains the analogue.

In one aspect, the analogue is an INSL3 B-chain analogue modified from the sequence set forth in SEQ ID NO:7.

In a further aspect, the INSL3 analogue is constrained by a cross-link between a first amino acid within a range of positions 2 and 8 and a second amino acid within a range of positions 21 and 26 of the sequences set forth in SEQ ID NO:7. Even more preferably, the INSL3 analogue is constrained by a cross-link between a first amino acid within a range of positions 2 and 8, including positions 2, 3, 4, 5, 6, 7, and 8, and a second amino acid within a range of positions 21 and 26, including 21, 22, 23, 24, 25 and 26, of the sequence set forth in SEQ ID NO:7.

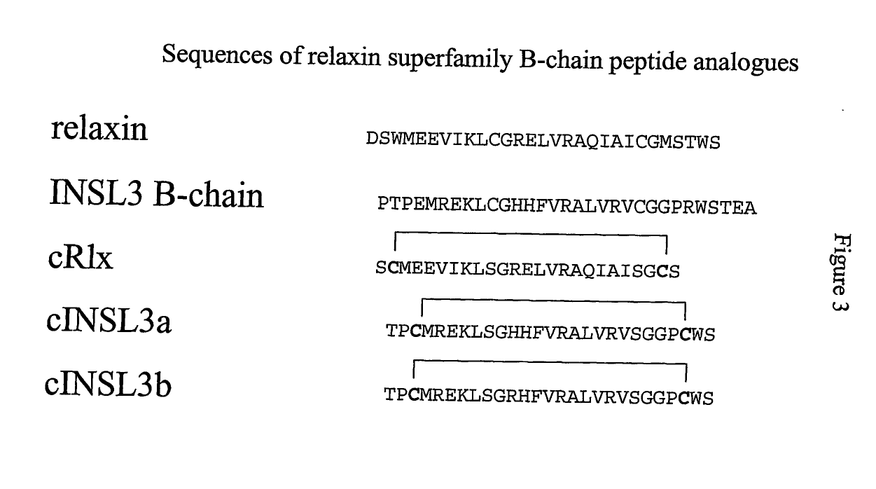

In a particularly preferred aspect, the B-chain peptide analogue is the INSL3 analogue designated cINSLa in FIG. 3.

In yet another preferred form, the B-chain peptide analogue is the INSL3 analogue designated cINSLb in FIG. 3.

In yet another aspect, the analogue is a relaxin analogue modified from a relaxin-1, relaxin-2, or relaxin-3 B-chain sequence set forth in SEQ ID NOs: 1, 2 and 3, respectively.

In a further aspect, the relaxin analogue is constrained by a cross-link between a first amino acid within a range of positions 2 and 8 and a second amino acid within a range of positions 21 and 26 of the sequences set forth in SEQ ID NO:2. Even more preferably, the relaxin analogue is constrained by a cross-link between a first amino acid within a range of positions 2 and 8, including positions 2, 3, 4, 5, 6, 7, and 8, and a second amino acid within a range of positions 21 and 26, including 21, 22, 23, 24, 25 and 26, of the sequence set forth in SEQ ID NO:2.

In a preferred form, the B-chain analogue is the relaxin analogue designated CR1x in FIG. 3.

In a particularly preferred form, the first and second amino acids are substituted with amino acids suitable for cross linking.

Suitable chemical linking groups have 0 to 20 carbon atoms, 0 to 10 heteroatoms (N, O, S, P etc.), and can be straight chain or branched an contain saturated, unsaturated and/or aromatic rings, single and/or double bonds and chemical groups such as amide, ester, disulfide, thioether, ether, phosphate, amine and the like.

The amino-acid substitution and formation of a cross-link, which is used to conformationally constrain the B-chain can be obtained by several methods, including but not limited to:

(i) cyclizing the N-terminal amine with the C-terminal carboxyl acid function, either directly via an amide bond between the N-terminal nitrogen and C-terminal carbonyl, or indirectly via a spacer group, for example by condensation with an M-amino carboxylic acid;

(ii) cyclizing via the formation of a covalent bond between the side chains of two residues, such as an amide bond between a lysine residue and either an aspartic acid or glutamic acid residue, or a disulfide bond between two cysteine residues, or a thioether bond between a cysteine residue and anz-halogenated amino acid residue, either directly or via a spacer group as described in (i) above. The residues contributing the side chains may be derived from the B-chain sequence itself, or may be incorporated into or added on to the B-chain sequence for this purpose; and,

(iii) cyclizing via the formation of an amide bond between a side chain (for example of a lysine or aspartate residue) and either the C-terminal carboxyl or N-terminal amine, either directly or using a spacer group as described in (i) above. The residues contributing the side chains may be derived from the B-chain sequence itself, or may be incorporated into or added on to the B-chain sequence for this purpose.

Furthermore, the B-chain peptide analogue can be made in which one or more amino acid residues is replaced by its corresponding D-amino acid, substitutions or modifications are made to one or more amino acids in the sequence, peptide bonds can be replaced by a structure more resistant to metabolic degradation and different cyclizing constraints and dimerization groups can be incorporated.

With respect to compounds in which one or more amino acids is replaced by its corresponding D-amino acid, the skilled person is aware that retro-inverso amino acid sequences can be synthesized by standard methods, see, for example, Chorev and Goodman, Acc. Chem. Res., 26: 266-273, 1993.

Olson et al., J. Med. Chem., 36: 3039-3049 1993 provides an example of replacing a peptide bond with a structure more resistant to metabolic degradation.

Analogues of the invention can also be made where individual amino acids are replaced by analogous structures, for example gem-diaminoalkyl groups or alkylmalonyl groups, with or without modified termini or alkyl, acyl or amine substitutions to modify their charge. The use of such alternative structures can provide significantly longer half-life in the body, since they are more resistant to breakdown under physiological conditions.

Methods for combinatorial synthesis of peptide analogues and for screening of peptides and peptide analogs are well known in the art (see, for example, Gallop et al., J. Med. Chem., 37: 1233-1251, 1994). It is particularly contemplated that the analogues of the invention are useful as templates for design and synthesis of compounds of improved activity, stability and bioavailability.

Preferably where amino acid substitution is used, the substitution is conservative, i.e. an amino acid is replaced by one of similar size and with similar charge properties.

As used herein, the term “conservative substitution” denotes the replacement of an amino acid residue by another, biologically similar residue. Examples of conservative substitutions include the substitution of one hydrophobic residue such as isoleucine, valine, leucine, alanine, cysteine, glycine, phenylalanine, proline, tryptophan, tyrosine, norleucine or methionine for another, or the substitution of one polar residue for another, such as the substitution of arginine for lysine, glutamic acid for aspartic acid, or glutamine for asparagine, and the like. Neutral hydrophilic amino acids which can be substituted for one another include asparagine, glutamine, serine and threonine. The term “conservative substitution” also includes the use of a substituted amino acid in place of an unsubstituted parent amino acid.

As such, it should be understood that in the context of the present invention, a conservative substitution is recognized in the art as a substitution of one amino acid for another amino acid that has similar properties. Exemplary conservative substitutions are set out in the following Table A from WO 97/09433.

TABLE A
CONSERVATIVE SUBSTITUTIONS I

| Side Chain Characteristic | Amino Acid |
|---|---|
| Aliphatic non-polar | G A P I L V |
| Polar - uncharged | C S T M N Q |
| Polar - charged | D E K R |
| Aromatic | H F W Y |
| Other | N Q D E |

Alternatively, conservative amino acids can be grouped as described in Lehninger, [Biochemistry, Second Edition; Worth Publishers, Inc. NY: NY (1975), pp. 71-77] as set out in the following Table B.

TABLE B
CONSERVATIVE SUBSTITUTIONS II

| Side Chain Characteristic | Amino Acid |
|---|---|
| Non-polar (hydrophobic) | |
| A. Aliphatic: | A L I V P |
| B. Aromatic: | F W |
| C. Sulphur-containing: | M |
| D. Borderline: | G |
| Uncharged-polar | |
| A. Hydroxyl: | S T Y |
| B. Amides | N Q |
| C. Sulfhydryl: | C |
| D. Borderline: | G |
| Positively Charged (Basic): | K R H |
| Negatively Charged (Acidic): | D E |

Exemplary conservative substitutions are set out in the following Table C.

TABLE C
CONSERVATIVE SUBSTITUTIONS III

| Original | |
|---|---|
| Residue | Exemplary Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe |
| Leu (L) | Ile, Val, Met, Ala, Phe |
| Lys (K) | Arg, Gln, Asn |
| Met (M) | Leu, Val, Ile, Ala |
| Pro (P) | Gly |
| Ser (S) | Thr |
| Thr (T) | Ser |
| Trp (W) | Tyr, Phe |
| Tyr (Y) | Trp, Phe, Thr, Ser |
| Val (V) | Ile, Leu, Met, Phe, Ala |

The cyclic peptide analogues of the invention can be modified, for instance, by glycosylation, amidation, carboxylation, or phosphorylation, or by the creation of acid addition salts, amides, esters, in particular C-terminal esters, and N-acyl derivatives of the peptides of the invention.

The peptide analogues also can be modified to create peptide derivatives by forming covalent or noncovalent complexes with other moieties. Covalently-bound complexes can be prepared by cross-linking the chemical moieties to functional groups on the side chains of amino acids comprising the peptides, or at the N-or C terminus.

The B-chain peptide analogues can be conjugated to an A-chain of a relaxin superfamily protein, and preferably, the A-chain of the relaxin superfamily protein to which they share B-chain analogy.

The B-chain peptide analogues can be conjugated to a reporter group, including, but not limited to a radiolabel, a fluorescent label, an enzyme (e.g., that catalyzes a colorimetric or fluorometric reaction), a substrate, a solid matrix, or a carrier (e.g., biotin or avidin).

In yet a further form, one or more amino acids within the peptide analogue sequence, other than the first and second amino acids, is optionally substituted to modify one or more biological activities of the analogue.

The term “biological activities” as used herein includes, but is not limited to, binding affinity for a biological target, effector activity at the biological target and in vivo stability.

In a further aspect, the invention seeks to provide a method of making a peptide analogue of a relaxin superfamily protein, the method comprising:
providing a B-chain, receptor-binding subunit of a relaxin superfamily protein; and
identifying a first and a second amino acid on respective, opposing strands of a turn and/or loop moiety in the receptor-binding subunit of the protein, with an alpha-helix or beta-strand carbon separation distance of less than six angstroms; and
introducing a cross-link between the first and second amino acids;
   wherein the cross-link constrains a three dimensional structure of at the B-chain, receptor-binding subunit.

In a particularly preferred form, the first and second amino acids are substituted with amino acids suitable for cross linking.

In one aspect, the method produces an INSL3 peptide analogue based on the sequence set forth in SEQ D) NO:7.

In a preferred form, the method produces an INSL3 analogue designated cR1x, cINSL3a or cINSL3b in FIG. 3.

In a further aspect, the method produces a relaxin-1, relaxin-2 or relaxin-3 peptide analogue based on any of the sequences set forth in SEQ ID NOs:1, 2 and 3, respectively.

Preferably, the method produces a relaxin analogue designated cR1x in FIG. 3.

In yet a further form, the method produces an INSL3 and/or relaxin analogue wherein one or more amino acids other than the cross-linked first and second amino acids, are optionally substituted to modify one or more biological activities of the peptide analogue.

One aspect of the invention utilises the ability of the B-chain peptide analogues of the invention to modulate an activity of a biological target of at least one relaxin superfamily protein member, that is, induce or modify an activity of at least one relaxin superfamily protein member, and in particular, relaxin or INSL3, which are mediated through the actions of the LGR7 or LGR8 receptors. The analogues can thus induce an activity of a relaxin superfamily member, in the absence of endogenous expression of the protein, or suppress or antagonise the activity of a protein endogenously expressed. Alternatively, the analogues of the invention can up-regulate the level of an existing biological activity of a relaxin superfamily member. Such biological activities include, but are not limited to, collagen degradation and remodelling, vascular dilation, growth and differentiation of the mammary gland, increased blood flow and organ perfusion, increasing heart rate, increasing coronory blood flow, increasing glomerular filtration rate and renal plasma flow, maturation of the gonads, promotion of cellular division, survival of sperm cells, development of ovarian follicles and oocyte maturation. As LGR8 is also expressed in the kidney, bone, peripheral blood leukoctyes, muscle, brain, thyroid, and uterus. Hence NSL3 analogues have potential actions on kidney and thyroid function, central actions on the brain, modulation of muscle function, effects on bone and blood cell function and reproductive actions on the uterus.

Accordingly, modification of any one or more of the above-mentioned biological activities of the relaxin superfamily proteins requires administration of an effective, biological-activity interfering amount of a B-chain analogue of the invention.

Clinical applications of the invention include, but are not limited to, hyperplastic disorders, neoplastic disorders, neurological disorders, angiogenic disorders, cardiovascular disorders, female reproductive disorders, conditions associated with pregnancy, renal disease, inflammatory bowel disease, Raynaud's disease, Raynaud's phenomenon, cryptorchidism, disregulation of spermatogenesis, male and female infertility and reproductive development including descent of the gonads.

In yet another aspect, there is provided a pharmaceutical composition including one or more of the analogues of the present invention, and pharmaceutically acceptable salts thereof.

Another aspect of the invention concerns the provision of a pharmaceutical composition comprising a B-chain analogue of the invention, and a pharmaceutically acceptable non-toxic salt thereof, and a pharmaceutically acceptable solid or liquid carrier or adjuvant. A preferred pharmaceutical composition will inhibit or interfere with a biological activity induced by at least one member of a relaxin superfamily protein. A particularly preferred pharmaceutical composition will inhibit the biological activity induced by relaxin and/or INSL3.

Examples of such a carrier or adjuvant include, but are not limited to, saline, buffered saline, Ringer's solution, mineral oil, talc, corn starch, gelatin, lactose, sucrose, microcrystalline cellulose, kaolin, mannitol, dicalcium phosphate, sodium chloride, alginic acid, dextrose, water, glycerol, ethanol, thickeners, stabilizers, suspending agents and combinations thereof. Such compositions may be in the form of solutions, suspensions, tablets, capsules, creams, salves, elixirs, syrups, wafers, ointments or other conventional forms.

The formulation is carried out to suit the mode of administration. Compositions comprising a peptide of the invention will contain from about 0.1% to 90% by weight of the active compound(s), and most generally from about 10% to 30%.

The dose(s) and route of administration will depend upon the nature of the patient and condition to be treated, and will be at the discretion of the attending physician or veterinarian.

Suitable routes include oral, subcutaneous, intramuscular, intraperitoneal or intravenous injection, parenteral, topical application, implants etc. For example, an effective amount of a monomeric monocyclic peptide of the invention or a dimeric bicyclic peptide of the invention is administered to an organism in need thereof in a dose between about 0.1 and 1000 μg/kg body weight.

The B-chain analogues of the invention may be used as therapeutic compositions either alone or in combination with other therapeutic agents.

Preferably, the pharmaceutical composition includes an INSL3 analogue or a relaxin analogue.

In one aspect, the disorder is characterised by disregulation of relaxin and/or INSL3 expression or activity.

As previously mentioned, it is understood that relaxin binds to at least two biological targets: LGR7 and LGR8, while INSL3 appears to mediate its biological effects via LGR8 only. Without being bound to any particular theory or mode of action, it is believed that an analogue with relaxin-like binding activity which exhibits affinity for LGR7 and LGR8 will modulate the effects of both relaxin and INSL3, given that INSL3 shares a common receptor with relaxin. Given the fact that LGR8 binds both relaxin and INSL3, it possible that many of relaxin's effects are modulated by INSL3 levels, and vice versa.

In another aspect, the invention provides a method of treating, preventing or ameliorating the symptoms of a relaxin or INSL3-mediated disorder in a patient, the disorder selected from the group consisting of hyperplastic disorders, neoplastic disorders, scleroderma, uncontrolled or abnormal collagen or fibronectin formation or breakdown, neurological disorders, angiogenic disorders, cardiovascular disorders, female reproductive disorders, conditions associated with pregnancy, renal disease, male and female infertility, inflammatory bowel disease, Raynaud's disease, Raynaud's phenomenon, cryptorchidism, disregulation of spermatogenesis and reproductive development including descent of the gonads, the method comprising administering to the patient an effective amount of a peptide of the invention.

As used herein, the term “conditions associated with pregnancy” includes, but is not limited to, conditions of fertilisation, pregnancy, parturition and lactation.

The term “relaxin or INSL3-mediated” as used herein refers to conditions where relaxin or INSL3 are involved or implicated in the development, maintenance or progression of the condition.

In a further aspect, the invention contemplates the use of the analogues and/or pharmaceutical compositions of the present invention in the manufacture of a medicament for the treatment of a relaxin or INSL3-mediated disorder in a patient, the disorder selected from the group consisting of: hyperplastic disorders, neoplastic disorders, scleroderma, neurological disorders, angiogenic disorders, cardiovascular disorders, female reproductive disorders, conditions associated with pregnancy, renal disease, male and female infertility, inflammatory bowel disease, Raynaud's disease, Raynaud's phenomenon, cryptorchidism, disregulation of spermatogenesis and reproductive development including descent of the gonads.

This invention will now be described with reference to the following, non-limiting examples.

### EXAMPLE 1

Methods and Materials

Molecular Design of INSL3 Analogues

Using SYBYL molecular modelling software (Tripos) on a Silicon Graphics O₂ workstation, we designed a model of INSL3 B-chain from the X-ray crystal structure of human Gene 2 relaxin B-chain as a template (Eigenbrot et al., 1991, J Mol Biol 221: 15-21). After modifying the sequence to resemble that of INSL3, an energy minimisation using a Tripos forcefield with Gasteiger-Marsili charges was carried out. From the resulting energy minimised model, two residues were identified (Glu⁴ and Arg²⁶) to have Cβ atoms within 4 Å of each other. These residues were then replaced with cysteine and a disulphide bond formed to give the cyclic peptide cINSLa.

A second cyclic analogue cINSLb was formed by changing His 12 to Arg, to mimic a relaxin-like binding motif of Arg-X-X-Arg-X-X-X-Val.

Solid-Phase Peptide Synthesis:

The synthesis of the INSL3 analogues were achieved using the continuous flow Fmoc-methodology as previously described (Dawson et al., J Peptide Res 53:542-547, 1999). The solid support was Fmoc-PAL PEG-PS (PerSeptive Biosystems, USA), and HBTU—activated Fmoc-amino acids were used throughout. N^{α}-Fmoc deprotection was with 20% piperidine in DMF. All derivatives were purchased from Auspep (Melbourne, Australia). Cleavage of the peptides from the solid support and side chain deprotection was achieved by a 3 hour treatment with trifluoroacetic acid (TFA) in the presence of phenol, thioanisole, ethanedithiol and water (82.5/5/5/2.5/5, v/v). The crude peptides were subjected to reversed-phase high performance liquid chromatography (RP-HPLC) on a Vydac C18 column (Hesperia, USA) using a 1%/min gradient of CH3CN in 0.1% aqueous TFA. Peptides were then oxidised in a buffer containing 10% dimethylsulfoxide (DMSO) for 24 hours and the reaction monitored on HPLC and by mass spectrometry.

Characterization:

The purity of the synthetic peptide was assessed by analytical RP-HPLC, and matrix-assisted laser desorption time of flight (MALDITOF) mass spectrometry using a Bruker Biflex instrument (Bremen, Germany) in the linear mode at 19.5 kV. Peptide quantitation was by amino acid analysis of a 24 hour acid hydrolyzate using a GBC instrument (Melbourne, Australia).

Circular Dichroism (CD) Spectroscopy:

CD spectra were taken on a Jasco J-720 instrument at room temperature in a 1 mm path-length cell, Doubly distilled water, 10 mM sodium phosphate buffer containing 120 mM NaCl pH 7.4, and spectroscopy grade trifluoroethanol (TFE) were used as solvents. The peptide concentrations were made to 1 μM, determined by quantitative RP-HPLC and amino acid analysis (ref). Curves were smoothed by the algorithm provided by Jasco, Mean residue ellipticity ([θ]_{MR}) is expressed in degrees×cm2/dmole by using the molecular mass. CD spectra evaluations were based on comparison with known peptide conformations (Otvos, L., Jr. 1996. from Neuropeptide protocols. Irvine, G. & Williams, C., Eds. pp 153-161. Humana Press, Totowa, N.J.).

Functional cAMP Assays:

Human 293T cells stably transfected with human LGR7, or LGR8 were preincubated in the presence of 0.25 mM 3-isobutyl-1-methyl xanthine (IBMX, Sigma) before various treatments for 30 minutes. At the end of the incubation cells were lysed for measurement of cAMP using a well-characterized cAMP ELISA (Satoko et al., J Biol Chem, 278:7855-7862). All experiments were repeated at least three times using cells from independent transfections, Results are plotted as pmol cAMP/10⁴ cells.

Ligand Binding Assays:

The method for binding of 33P-labelled relaxin to 293T cells stably transfected with either LGR7 or LGR8 has been described previously (Satoko et al., J Biol Chem, 278:7855-7862). Data are expressed as mean±SEM of % specific binding of triplicate determinations performed on a least three independent plates of transfected cells. Data were plotted using the one-site competition functions of the PRISM program (Graphpad Inc., San Diego, USA).

### EXAMPLE 2

Sites for the incorporation cyclising constraints to create B chain analogues were determined by molecular modelling studies. In the case of relaxin, distances between β-carbon atoms in the B chain were measured in the X-ray crystal structure of relaxin [6RLX], using the molecular modelling program Sybyl (Tripos Inc St Louis Mo.). Residue 25 (Met) within the C chain helix and residue 3 (Trp) in the strand were found to have a β-carbon to β-carbon distance of 3.855 angstroms. These residues were thus deemed suitable for replacement by Cys and subsequent formation of a disulphide bond. This constrained two ends of the molecule in a similar fashion to the native protein, resulting in the cyclic relaxin B chain mimetic cR1x (FIG. 3). Methods are as described in International Patent Applications Nos. WO 00/75176 and WO 01/52875.

In the case of INSL3 we constructed a model of the 3D structure of INSL3 by substituting the appropriate amino acids into the relaxin structure, and carrying out an energy minimisation of the resultant INSL3 B chain. We then identified pairs of residues as potential sites for the incorporation of a Cys to Cys constraint, including the pair Glu4 and Arg26, residues adjacent to those replaced in the relaxin analogue, giving the cyclic INSL3 mimetic cINSLa (FIG. 3).

Following the biological characterisation of peptides cR1x and cINSLa (FIG. 3), an additional compound was prepared incorporating the Cys to Cys constraint of peptide cINSLa. This compound, designated cINSLb, was a INSL3-based sequence, in which His12 form the INSL3 sequence was replaced by Arg in an attempt to obtain an analogue of the putative relaxin receptor binding cassette which if absent in INSL3.

The same approach is used to prepare insulin B chain analogues and other relaxin superfamily analogues.

Although several preferred embodiments have been described in detail, it should be understood that various changes, substitutions, and alterations can be made herein by one ordinarily skilled in the art without departing from the spirit or scope of the present invention.

TABLE 1

SEQ
ID

| | |
|---|---|
| NO: | RELAXIN SUPERFAMILY B-CHAIN PEPTIDE/ANALOGUE |

| | |
|---|---|
| 1 | relaxin-1 b-chain |
| 2 | relaxin-2 b-chain |
| 3 | relaxin 3 b-chain |
| 4 | insulin b-chain |
| 5 | IGF-1 b-chain |
| 6 | IGF-2 b-chain |
| 7 | INSL3 b-chain |
| 8 | INSL4 b-chain |
| 9 | INSL5 b-chain |
| 10 | INSL6 b-chain |
| 11 | cyclic relaxin b-chain mimetic |
| 12 | INSL3 b-chain based peptide analogue 4 |
| 13 | INSL3 b-chain based peptide analogue 5 |
| 14 | INSL3 b-chain based peptide analogue 6 |
| 15 | INSL3 b-chain based peptide analogue 7 |
| 16 | relaxin-1 a-chain |
| 17 | relaxin-2 a-chain |
| 18 | retaxin-3 a-chain |
| 19 | insulin a-chain |
| 20 | IGF-1 a-chain |
| 21 | IGF2 a-chain |
| 22 | INSL3 a-chain (Ley I-L/RLF) |
| 23 | INSL4 a-chain (placentin/EPIL) |
| 24 | INSL5 a-chain |
| 25 | INSL6 a-chain |

1. A monomeric, cyclic peptide analogue of a B-chain of a relaxin superfamily member protein which binds to a biological target of the relaxin superfamily protein, and modulates an activity of the biological target, wherein the relaxin superfamily protein is selected from insulin, IGF-I, IGF-II, relaxin 1, relaxin 2, relaxin 3, INSL3, INSL4, INSL5 and INSL6, the biological target being selected from insulin receptors, IGFR-I, IGFR-II, LGR7 and LGR8.

2. The analogue according to claim 1, wherein the analogue is produced by modification of a turn or loop moiety of the B-chain of the relaxin superfamily protein, the modification involving selection of at least a first and a second amino acid residue with an alpha-helix or beta-strand carbon separation distance of less than six angstroms and cross-linking the first and second amino acids, wherein the cross-link conformationally constrains the analogue.

3. The analogue according to claim 1 or claim 2, wherein the analogue is an INSL3 B-chain analogue modified from a sequence set forth in SEQ ID NO:7.

4. The analogue according to claim 3, wherein the INSL3 analogue is constrained by a cross-link between a first amino acid within a range of positions 2 and 8 and a second amino acid within a range of positions 21 and 26 of the sequence set forth in SEQ ID NO:7.

5. The analogue according to any one claims 3 to 4, wherein the analogue is the INSL3 analogue designated cINSL3a in FIG. 3.

6. The analogue according to any one of claims 3 to 4, wherein the analogue is the INSL3 analogue designated cINSL3b in FIG. 3.

7. The analogue according to claim 1 or claim 2, which is a relaxin analogue modified from a relaxin-1, relaxin-2, or relaxin-3 B-chain sequence set forth in SEQ ID NOs:1, 2 and 3, respectively.

8. The analogue according to claim 7, wherein the relaxin analogue is constrained by a cross-link between a first amino acid within a range of positions 2 and 8 and a second amino acid within a range of positions 21 and 26 of the sequence set forth in SEQ ID NO:2.

9. The analogue according to claim 8, wherein the relaxin analogue is the relaxin analogue designated cR1x in FIG. 3.

10. The analogue according to claim 2, wherein the first and/or second amino acids are substituted with alternative amino acids suitable for cross-linking.

11. The analogue according to claim 10 wherein at least one of the alternative amino acids is a cysteine residue.

12. The analogue according to claim 11 wherein both of the alternative amino acid residues are cysteine residues.

13. The analogue according to claim 12 wherein the analogue is cross-linked by oxidizing the cysteine residues to form a disulfide bond between the cysteine residues.

14. An analogue according to any one of claims 1 to 13, wherein one or more amino acids within the INSL3 or relaxin peptide analogue sequence, other than the cross-linked first and second amino acids, is optionally substituted to modify one or more biological activities of the analogue.

15. The analogue according to any one of claims 1 to 14, wherein the biological target of the analogue is LGR7 and/or LGR8.

16. The analogue according to claim 15, wherein activity of the biological target is initiated, up-regulated, down-regulated or otherwise blocked.

17. The analogue of any of the preceding claims, wherein the analogue is conjugated to an A-chain of a relaxin superfamily protein.

18. The analogue according to claim 17, wherein the A-chain of the relaxin superfamily protein is a corresponding A-chain for the relaxin superfamily protein.

19. The analogue according to any one of claims 1 to 18, wherein the analogue is conjugated to a reporter group.

20. The analogue according the claim 19, wherein the reporter group is a radiolabel.

21. The analogue according to claim 19, wherein the reporter group is a fluorescent label.

22. The analogue according to claim 19, wherein the reporter group is an enzyme.

23. The analogue according to claim 19, wherein the reporter group is a carrier.

24. A method of making a peptide analogue of a relaxin superfamily protein, the method comprisin:
providing a B-chain, receptor-binding subunit of a relaxin superfamily protein; and
identifying a first and a second amino acid on respective, opposing strands of a turn and/or loop moiety in the receptor-binding subunit of the protein, with an alpha-helix or beta-strand carbon separation distance of less than six angstroms; and
introducing a cross-link between the first and second amino acids;
wherein the cross-link conformationally constrains a three dimensional structure of at the B-chain, receptor-binding subunit.

25. The method according to claim 24, wherein the first and/or second amino acids are substituted with amino acids suitable for cross-linking.

26. The method according to claim 24 or 25, wherein the method produces an INSL3 peptide analogue based on the sequence set forth in SEQ ID NO:7, with the first amino acid within a range of residues 2 and 8 and the second amino acid within a range of residues 21 and 26.

27. The method according to claim 26, wherein the INSL3 analogue is cINSL3a as designated in FIG. 3.

28. The method according to claim 26, wherein the INSL3 analogue is cINSL3b as designated in FIG. 3.

29. The method according to claim 24 or 25, wherein the method produces a relaxin-2 peptide analogue based on the sequence set forth in SEQ ID NO:2, with the first amino acid within a range of residues 2 and 8 and the second amino acid within a range of residues 21 and 26.

30. The method according to claim 29, wherein the method produces the relaxin analogue designated cR1x in FIG. 3.

31. The method according to any one of claims 24 to 26 or 29, wherein the method produces an INSL3 or relaxin analogue wherein one or more amino acids other than the cross-linked first and second amino acids, are optionally substituted to modify one or more biological activities of the peptide analogue.

32. A pharmaceutical composition including one or more of the analogues as defined in any one of claims 1 to 23, or pharmaceutically acceptable salts thereof.

33. The pharmaceutical compositions according to claim 32, further comprising at least one pharmaceutically acceptable carrier or diluent.

34. A method of treating, preventing or ameliorating the symptoms of a disorder in a patient, the disorder involving disregulation of activity of a relaxin superfamily protein member, the method comprising administering to the patient an effective amount of a peptide analogue according to any one of claims 1 to 23.

35. The method according to claim 34, wherein the disorder is mediated by relaxin or INSL3.

36. The method according to claim 35, wherein the disorder selected from the group consisting of hyperplastic disorders, scleroderma, uncontrolled or abnormal collagen or fibronectin formation or breakdown, neoplastic disorders, neurological disorders, angiogenic disorders, cardiovascular disorders, female reproductive disorders, conditions associated with pregnancy, renal disease, inflammatory bowel disease, Raynaud's disease, Raynaud's phenomenon, cryptorchidism, male or female infertility, disregulation of spermatogenesis and reproductive development including descent of the gonads, the method comprising administering to the patient an effective amount of a peptide analogue according to any one of claims 1 to 23, or a pharmaceutical composition according to any one of claims 32 to 33.

37. The method according to claim 36, wherein the disorder is a female reproductive disorder.

38. The method according to claim 36, wherein the disorder is a pregnancy-related disorder.

39. The method according to claim 36, wherein the disorder involves abnormal reproductive development.

40. The method according to claim 36, wherein the disorder is a cardiovascular disorder.

41. The method according to claim 36, wherein the disorder is an angiogenic disorder.

42. The method according to claim 36, wherein the disorder is a neoplastic disorder or a hyperplastic disorder.

43. The method according to claim 42, wherein the neoplastic or hyperplastic disorder involves the thyroid gland.

44. The method according to claim 36, wherein the disorder is scleroderma.

45. The method according to claim 36, wherein the disorder is renal disease.

46. The method according to claim 36, wherein the disorder involves uncontrolled or abnormal collagen or fibronectin formation or breakdown.

47. The method according to claim 36, wherein the disorder is male or female infertility.

48. Use of the analogues and/or pharmaceutical compositions of the present invention in the manufacture of a medicament for the treatment of a relaxin or INSL3-mediated disorder in a patient.

49. Use according to claim 48, wherein the disorder is selected from the group consisting of: hyperplastic disorders, neoplastic disorders, scleroderma, uncontrolled or abnormal collagen or fibronectin formation or breakdown, neurological disorders, angiogenic disorders, cardiovascular disorders, female reproductive disorders, conditions associated with pregnancy, renal disease, inflammatory bowel disease, Raynaud's disease, Raynaud's phenomenon, cryptorchidism, disregulation of spermatogenesis and reproductive development including descent of the gonads.
